(19) United States
(12) Patent Application Publication
(10) Pub. No.: US 2014/0276925 A1
(43) Pub. Date: Sep. 18, 2014

(54) METHODS AND SYSTEMS FOR USE IN GUIDING IMPLANTATION OF A NEUROMODULATION LEAD

(71) Applicant: SPINAL MODULATION, INC., Menlo Park, CA (US)

(72) Inventors: Jeffrey M. Alves, Pleasanton, CA (US); Jeyakumar Subbaroyan, Menlo Park, CA (US); Jeffery M. Kramer, San Francisco, CA (US); Lynn Elliott, Maple Grove, MN (US)

(73) Assignee: SPINAL MODULATION, INC., Menlo Park, CA (US)

(21) Appl. No.: 13/795,931

(22) Filed: Mar. 12, 2013

(51) Int. Cl. A61N 1/05

(52) U.S. Cl. CPC A61N 1/0551
USPC 606/129

(57) ABSTRACT

Methods and systems for use in guiding implantation of a neuromodulation lead during an implant procedure are disclosed herein. Certain methods and system are for use in guiding implantation of a lead toward an implant position where at least one electrode of the lead is located near a target dorsal root ganglion (DRG). Such methods can involve inserting a distal end of the lead into an epidural space of a spinal column within which is located the target DRG, and using one or more electrodes of the lead to obtain a sensed signal indicative of proximity of at least one electrode of the lead relative to the target DRG. Additionally, the sensed signal is used to guide placement of at least one electrode of the lead toward the implant position near the target DRG.

### FIELD OF THE INVENTION

Embodiments of the present invention generally relate to methods and systems for use in guiding implantation of a neuromodulation lead during an implant procedure.

### BACKGROUND OF THE INVENTION

It has recently been discovered that directly neuromodulating one or more target dorsal root ganglion (DRG) can be used to treat various types of conditions associated with or influenced by the nervous system. Such neuromodulating often involves stimulating one or more target DRG for the purpose of treating pain. However, other types of conditions that can be treated by stimulating target DRG include itching, Parkinson's Disease, Multiple Sclerosis, movement disorders, spinal cord injury, asthma, chronic heart failure, obesity and stroke (particularly acute ischemia), to name a few. Further, pharmaceutical agents can alternatively, or additionally, be used to perform the neuromodulating of target DRG.

In order to deliver stimulation to a target DRG, a lead including one or more electrodes should be implanted in a patient such that an electrode of the lead is located in close proximity to the target DRG.

Imaging techniques, such as fluoroscopy, are often used by a physician to help guide the lead during implant of the lead. While imaging techniques have enabled physicians to successfully implant leads that are used to stimulate target DRG, it would be beneficial if additional techniques were available for aiding, improving and preferably optimizing the guiding of leads during implantation.

### BRIEF SUMMARY

Embodiments of the present invention described herein generally relate to methods and systems for use in guiding implantation of a neuromodulation lead during an implant procedure. Certain methods are for use in guiding implantation of a lead toward an implant position where at least one electrode of the lead is located near a target dorsal root ganglion (DRG). Such methods can involve inserting a distal end of the lead into an epidural space of a spinal column within which is located the target DRG, and using at least one electrode of the lead to obtain a sensed signal indicative of proximity of the at least one electrode relative to the target DRG. For example, the sensed signal can be obtained while the distal end of the lead is advanced toward or otherwise maneuvered relative to the target DRG. Additionally, the sensed signal is used to guide placement of at least one electrode of the lead toward the implant position near the target DRG. The term near, as used herein, means as close as reasonably possible to the target DRG, including on (i.e., touching), about or adjacent the target DRG, but in general means within about 10 mm of the target DRG, preferably within about 5 mm of the target DRG, and more preferably within about 1-2 mm of the target DRG.

The sensed signal can be an intrinsic signal. Alternatively, the sensed signal can be an evoked response signal. For example, if the lead being implanted is for use in stimulating the target DRG to treat pain in a targeted body region, such as the patient's right foot, then physical or electrical stimulation can be delivered to the patient's right foot to induce an evoked response signal that is sensed and used to guide placement of at least one electrode of the lead toward the implant position near the target DRG.

In accordance with certain embodiments, the sensed signal is indicative of a potential difference between an electrode of the lead and a further electrode, wherein the further electrode can also be part of the lead, or alternatively, the further electrode can be a far-field reference electrode, such as, but not limited to, a patch electrode. In such embodiments, the sensed signal, which can also be referred to as a sensed voltage signal, is used to guide placement of at least one electrode of the lead toward an implant position near the target DRG. This can involve interpreting an increase in the sensed voltage signal as an indication that at least one electrode, used to obtain the sensed voltage signal, moved closer to the target DRG, and/or interpreting a decrease in the sensed voltage signal as an indication that at least one electrode, used to obtain the sensed voltage signal, moved away from the target DRG. Alternatively, or additionally, a signal-to-noise ratio (SNR) of the sensed voltage signal can be determined, and an increase in the SNR can be interpreted as an indication that at least one electrode, used to obtain the sensed voltage signal, moved closer to the target DRG. Conversely, a decrease in the SNR can be interpreted as an indication that at least one electrode, used to obtain the sensed voltage signal, moved away from the target DRG.

In accordance with certain embodiments, the sensed signal is indicative of the impedance between an electrode of the lead and a further electrode, wherein the further electrode can also be part of the lead, or alternatively, the further electrode can be a far-field reference electrode, such as, but not limited to, a patch electrode. In such embodiments, the sensed signal, which can also be referred to as a sensed impedance signal, is used to guide placement of at least one electrode of the lead toward an implant position near the target DRG. This can involve interpreting an increase in the sensed impedance signal as an indication that at least one electrode, used to obtained the sensed impedance signal, moved closer to the target DRG.

In accordance with an embodiment, an audio indicator (e.g., a tone or other sound) is produced in dependence on the sensed signal. In such an embodiment, changes in an amplitude, frequency and/or repetition rate of the audio indicator are indicative of changes in proximity of at least one electrode of the lead relative to the target DRG. Alternatively, or additionally, a visual indicator (e.g., a bar graph) is produced in dependence on the sensed signal, in which case changes in the visual indicator (e.g., changes in the height of a bar graph) are indicative of changes in proximity of at least one electrode of the lead relative to the target DRG. Alternatively, or additionally, a tactile indicator (e.g., a vibration) is produced in dependence on the sensed signal, in which case changes in the tactile indicator (e.g., changes in the magnitude or frequency of vibration) are indicative of changes in proximity of at least one electrode of the lead relative to the target DRG.

In alternative embodiments, a sense element used to obtain the sensed signal need not be an electrode of the lead that is being implanted. Rather, a sense element can be an electrode or other type of sensor (e.g., a chemical sensor) that is part of a sheath that is being used during the implant procedure. In such embodiments, the sensed signal can be used to guide positioning of the sense element to a position near a target DRG.

Certain embodiments of the present invention relate to systems for use in guiding implantation of a lead toward an implant position where at least one electrode of the lead is located near a target DRG. In accordance with an embodiment, such a system includes circuitry to obtain a sensed signal from a lead after a distal end of the lead has been inserted into an epidural space of a spinal column within which is located the target DRG. The system also includes a transducer that produces an indicator in dependence on the sensed signal, wherein changes in the indicator are indicative of changes in proximity of at least one electrode of the lead relative to the target DRG. The system can also include a controller coupled between the circuitry to obtain the sensed signal and the transducer. Such a controller can interpret changes in the sensed signal and adjust the indicator so that changes in the indicator are indicative of changes in the proximity of at least one electrode of the lead relative to the target DRG. In an embodiment, the transducer is an audio transducer (e.g., a speaker) that produces an audio indicator (e.g., a tone or other sound) that is controlled by the controller. For example, the controller can change the amplitude, frequency and/or repetition rate of a beeping sound to indicate changes in the proximity of at least one electrode of the lead to the target DRG. A visual transducer that produces a visual indicator and/or a tactile transducer that produces a tactile indicator can alternatively or additionally be included in the system. Such a system can include a single device, or can be made up of multiple devices. For example, such a system can include a trial neurostimulation (TNS) device and a clinical programmer device that communicate with one another, but is not limited thereto.

This summary is not intended to summarize all of the embodiments of the present invention. Further and alternative embodiments, and the features, aspects, and advantages of the embodiments of invention will become more apparent from the detailed description set forth below, the drawings and the claims.

### BRIEF DESCRIPTION OF THE DRAWINGS

FIG. 1 illustrates an exemplary embodiment of a lead advanced through a nerve root sleeve angulation so that at least one of its electrodes is positioned near a target DRG.

FIGS. 2A, 2B, 2C, 2D illustrate an exemplary embodiment of a lead and delivery system, including a sheath, stylet and introducing needle.

FIG. 3 illustrates an exemplary embodiment of a sheath advanced over a shaft of a lead with internal stylet forming a first curvature.

FIG. 4 illustrates the lead with internal stylet of FIG. 3 extending beyond the sheath forming a second curvature.

FIG. 5 illustrates a method of accessing an epidural space with the use of an introducing needle.

FIG. 6 illustrates a method of attaching a syringe to the needle of FIG. 5.

FIG. 7 illustrates a method of inserting a stylet, lead and sheath through the needle of FIG. 5 into the epidural space.

FIG. 8 illustrates the distal end of the needle passed through the ligamentum flavum into the epidural space and the assembled sheath/lead/stylet of FIG. 7 emerging therefrom.

FIG. 9 illustrates advancing the assembled sheath/lead/stylet of FIG. 7 within the epidural space toward a target DRG.

FIG. 10 illustrates the precurvature of the sheath directing the lead laterally outwardly.

FIG. 11 illustrates the lead extending beyond the distal end of the sheath of FIG. 10.

FIG. 12 illustrates a method of using the needle of FIG. 5 to position an additional lead within the epidural space.

FIG. 13 illustrates an additional assembled sheath/lead/stylet advanced within the epidural space toward another or second target DRG.

FIG. 14 illustrates the precurvature of the sheath of FIG. 13 directing the lead laterally outwardly.

FIG. 15 illustrates the lead advanced beyond the distal end of the sheath of FIG. 14.

FIG. 16 illustrates a plurality of leads positioned within the epidural space, each lead stimulating a different DRG.

FIG. 17 is a high level flow diagram that is used to summarize various methods for use in guiding implantation of a lead toward an implant position where an electrode of the lead is located near a target DRG.

FIG. 18 is a high level block diagram that illustrates components of a non-implanted lead implant guidance (LIG) device, according to an embodiment of the present invention.

FIG. 19 illustrates an exemplary visual transducer that can be part of the LIG device of FIG. 18.

### DETAILED DESCRIPTION

The following description is of various embodiments of the present invention. The description is not to be taken in a limiting sense but is made merely for the purpose of describing the general principles of the invention. The scope of the invention should be ascertained with reference to the claims. In the description of the invention that follows, like numerals or reference designators will be used to refer to like parts or elements throughout.

As mentioned above, imaging techniques, such as fluoroscopy, are often used by a physician to help guide a lead during implant of the lead that is going to be used to neuromodulate a target DRG. As also mentioned above, while imaging techniques have enabled physicians to successfully implant leads that are used to stimulate target DRG, it would be beneficial if additional techniques were available for aiding, improving and preferably optimizing the guiding of leads during implantation. Such additional techniques can be used to supplement, or potentially replace, imaging techniques that are currently being used. Embodiments of the present invention, which are described herein, generally relate to additional methods, devices and systems for use in guiding implantation of a neuromodulation lead during an implant procedure.

In specific embodiments, the methods, devices and systems are used to obtain a sensed signal indicative of proximity of an electrode relative to a target DRG, and the sensed signal is used to guide placement of an electrode of the lead toward an implant position near the DRG. The term near, as used herein, means as close as reasonably possible to the target DRG, including on (i.e., touching), about or adjacent the target DRG, but in general means within about 10 mm of the target DRG, preferably within about 5 mm of the target DRG, and more preferably within about 1-2 mm of the target DRG. Additional details of such embodiments are provided below. However, prior to providing these additional details it would first be useful to provide details of exemplary neuromodulation leads, as well as exemplary lead delivery systems that are used during the implant procedure. For completeness, additional details of the DRG, including its location, function and the anatomy in its vicinity, are also provided below.

The central nervous system includes the spinal cord and the pairs of nerves along the spinal cord which are known as spinal nerves. The spinal nerves include both dorsal and ventral roots which fuse in the intravertebral foramen to create a mixed nerve which is part of the peripheral nervous system. At least one DRG is disposed along each dorsal root prior to the point of mixing. Thus, the neural tissue of the central nervous system is considered to include the dorsal root ganglions and exclude the portion of the nervous system beyond the dorsal root ganglions, such as the mixed nerves of the peripheral nervous system.

Implanting a lead so that at least one electrode of the lead is positioned near a target DRG may be challenging. This is because accessing the DRG may be challenging, particularly if accessed from an antegrade epidural approach. FIG. 1 schematically illustrates portions of the anatomy in such areas. As shown, each DRG is disposed along a dorsal root DR and typically resides at least partially between the pedicles PD or within a foramen. Each dorsal root DR exits the spinal cord S at an angle θ. This angle θ is considered the nerve root sleeve angulation and varies slightly by patient and by location along the spinal column. The average nerve root angulation in the lumbar spine is significantly less than 90 degrees and typically less than 45 degrees. Therefore, accessing this anatomy from an antegrade approach involves making a sharp turn through, along or near the nerve root sleeve angulation. It may be appreciated that such a turn may follow the nerve root sleeve angulation precisely or may follow various curves in the vicinity of the nerve root sleeve angulation.

FIG. 1 illustrates an exemplary lead 100 inserted epidurally and advanced in an antegrade direction along the spinal cord S. The lead 100, having at least one electrode 102 thereon, is advanced through the patient anatomy so that at least one of the electrodes 102 is positioned on a target DRG. Such advancement of the lead 100 toward the target DRG in this manner involves making a sharp turn along the angle θ. A turn of this severity can be achieved, for example, using delivery tools and design features described in commonly assigned U.S. patent application Ser. No. 12/687,737, which has been published as US 2010/0179652 A1, and which is incorporated herein by reference. In addition, the spatial relationship between the nerve roots, DRGs and surrounding structures are significantly influenced by degenerative changes, particularly in the lumbar spine. Thus, patients may have nerve root angulations which differ from the normal anatomy, such as having even smaller angulations necessitating even tighter turns.

Optimal placement of the lead 100, and its electrode(s) 102, allows for targeted treatment of the desired anatomies. Such targeted treatment minimizes deleterious side effects, such as undesired motor responses or undesired stimulation of unaffected body regions. This is achieved by directly neuromodulating a target anatomy associated with the condition while minimizing or excluding undesired neuromodulation of other anatomies. For example, this may include stimulating the target DRG while minimizing or excluding undesired stimulation of other tissues, such as surrounding or nearby tissues, portions of the ventral root and portions of the anatomy associated with body regions which are not targeted for treatment.

During implantation of the lead 100, the lead 100 is advanced through the patient anatomy so that the at least one electrode 102 is positioned near the target DRG. As mentioned above, guidance of the lead 100 during implantation has typically been performed using imaging techniques, such as fluoroscopy.

### Exemplary Lead and Related Delivery System

FIGS. 2A-2D will now be used to illustrate details of an exemplary lead 100 (FIG. 2A) and an exemplary delivery system 120, including a sheath 122 (FIG. 2B), stylet 124 (FIG. 2C) and introducing needle 126 (FIG. 2D). In this embodiment, the lead 100 comprises a shaft 103 having a distal end 101 and four electrodes 102 disposed thereon. It may be appreciated that any number of electrodes 102 may be present, including one, two, three, four, five, six, seven, eight or more. In this example, the distal end 101 has a closed-end distal tip 106. The distal tip 106 may have a variety of shapes including a rounded shape, such as a ball shape (shown) or tear drop shape, and a cone shape, to name a few. These shapes provide an atraumatic tip for the lead 100 as well as serving other purposes. The lead 100 also includes a stylet lumen 104 which extends toward the closed-end distal tip 106.

FIG. 2B illustrates an exemplary embodiment of a sheath 122. The sheath 122 has a distal end 128 which is pre-curved to have an angle α, wherein the angle α is in the range of approximately 80 to 165 degrees. The sheath 122 is sized and configured to be advanced over the shaft 103 of the lead 100 until a portion of its distal end 128 abuts the distal tip 106 of the lead 100, as illustrated in FIG. 3. Thus, the ball shaped tip 106 also prevents the sheath 122 from extending thereover. Passage of the sheath 122 over the lead 100 causes the lead 100 to bend in accordance with the precurvature of the sheath 122. Thus, the sheath 122 assists in steering the lead 100 along the spinal column S and toward a target DRG, such as in a lateral direction. It may be appreciated that the angle α may optionally be smaller, such as less than 80 degrees, forming a U-shape or tighter bend.

Referring back to FIG. 2C, an exemplary embodiment of a stylet 124 is illustrated. The stylet 124 has a distal end 130 which is pre-curved so that its radius of curvature is in the range of approximately 0.1 to 0.5 inches. The stylet 124 is sized and configured to be advanced within the stylet lumen 104 of the lead 100. Typically the stylet 124 extends therethrough so that its distal end 130 aligns with the distal end 101 of the lead 100. Passage of the stylet 124 through the lead 100 causes the lead 100 to bend in accordance with the precurvature of the stylet 124. Typically, the stylet 124 has a smaller radius of curvature, or a tighter bend, than the sheath 122. Therefore, as shown in FIG. 4, when the stylet 124 is disposed within the lead 100, extension of the lead 100 and stylet 124 through the sheath 122 bends or directs the lead 100 through a first curvature 123. Further extension of the lead 100 and stylet 124 beyond the distal end 128 of the sheath 122 allows the lead 100 to bend further along a second curvature 125. When approaching a target DRG, the second curvature allows the laterally directed lead 100 to now curve around toward the target DRG, such as along the nerve root angulation. This two step curvature allows the lead 100 to be successfully positioned so that at least one of the electrodes 102 is near the target DRG, particularly by making a sharp turn along the angle θ. In addition, the electrodes 102 are spaced to assist in making such a sharp turn.

Thus, the lead 100 does not require stiff or torqueable construction since the lead 100 is typically not torqued or steered by itself. The lead 100 is positioned with the use of the sheath 122 and stylet 124 which direct the lead 100 through the two step curvature. This eliminates the need for the operator to torque the lead 100 and optionally the sheath 122 with multiple hands. This also allows the lead 100 to have a lower profile and smaller diameter, as well as a very soft and flexible construction. This, in turn, minimizes erosion, irritation of the neural tissue and discomfort created by pressure on nerve tissue, such as the target DRG and/or the nerve root, once the lead 100 is implanted. In addition, such a soft and flexible lead 100 will minimize the amount of force translated to the distal end of the lead 100 by body movement (e.g. flexion, extension, torsion).

Referring back to FIG. 2D, an exemplary implementation of an introducing needle 126 is illustrated. The introducing needle 126 is used to access the epidural space of the spinal cord S. The needle 126 has a hollow shaft 127 and typically has a very slightly curved distal end 132. The shaft 127 is sized to allow passage of the lead 100, sheath 122 and stylet 124 therethrough. In some implementations, the needle 126 is 14 gauge which is typically the size of epidural needles used to place conventional percutaneous leads within the epidural space. However, it may be appreciated that other sized needles may also be used, particularly smaller needles such as 15-18 gauge. Alternatively, non-standardized sized needles may be used.

The needle is preferably atraumatic so as to not damage the sheath 122 when the sheath 122 is advanced or retracted. In some implementations, the shaft 127 comprises a low friction material, such as bright hypotubing, made from bright steel (a product formed from the process of drawing hot rolled steel through a die to impart close dimensional tolerances, a bright, scale free surface and improved mechanical properties. Other materials include polytetrafluoroethylene (PTFE) impregnated or coated hypotubing. In addition, it may be appreciated that needles having various tips known to practitioners or custom tips designed for specific applications may also be used. The needle 126 also typically includes a luer fitting 134, such as a Luer-Lok™ fitting, or other fitting near its proximal end. The luer fitting 134 is a female fitting having a tabbed hub which engages threads in a sleeve on a male fitting, such as a syringe. The needle 126 may also have a luer fitting on a side port, so as to allow injection through the needle 126 while the sheath 122 is in the needle 126. In some implementations, the luer fitting is tapered to allow for easier introduction of a curved sheath into the hollow shaft 127.

Additional details of the above described exemplary lead 100 and delivery system 120 are provided in commonly assigned U.S. patent application Ser. No. 12/687,737, which has been published as US 2010/0179652 A1, and which was incorporated herein by reference above. Further, it should be noted that while the exemplary lead 100 and delivery system 120 have been described herein, embodiments of the present invention are not intended to be limited to use with this specific lead 100 and delivery system 120.

### Exemplary Lead Implantation Methods

As mentioned above, embodiments of the present invention generally relate to methods, devices and systems for use in guiding implantation of a neuromodulation lead (e.g., lead 100) during an implant procedure. Accordingly, before describing such embodiments, exemplary implant procedures, with which embodiments of the present invention may be used, are first described.

Lead implant procedures are sometimes also referred to as delivery methods, since they are used to deliver a lead to a desired position. For example, the above described delivery system 120 can be used for epidural delivery of the lead 100 through the patient anatomy toward a target DRG. Thus, exemplary epidural delivery methods are described herein, which can involve an antegrade approach, or alternatively, a retrograde approach or a contralateral approach. It would also be possible to use a transforaminal approach, wherein the DRG is approached from outside of the spinal column. Further, the target DRG may be approached through the sacral hiatus or through a bony structure such as a pedicle, lamina or other structure.

Epidural delivery involves accessing the epidural space. The epidural space is accessed with the use of the introducing needle 126, as illustrated in FIG. 5. Typically, the skin is infiltrated with local anesthetic such as lidocaine over the identified portion of the epidural space. The insertion point is usually near the midline M, although other approaches may be employed. Typically, the needle 126 is inserted to the ligamentum flavum and a loss of resistance to injection technique is used to identify the epidural space. Referring to FIG. 6, a syringe 140 is then attached to the needle 126. The syringe 140 may contain air or saline. Traditionally either air or saline has been used for identifying the epidural space, depending on personal preference. When the tip of the needle 126 enters a space of negative or neutral pressure (such as the epidural space), there will be a “loss of resistance” and it will be possible to inject through the syringe 140. At that point, there is now a high likelihood that the tip of the needle 126 has entered the epidural space. Further, a sensation of “pop” or “click” may be felt as the needle breaches the ligamentum flavum just before entering the epidural space. In addition to the loss of resistance technique, realtime observation of the advancing needle 126 may be achieved with a portable ultrasound scanner or with fluoroscopy. Optionally, a guidewire may be advanced through the needle 126 and observed within the epidural space with the use of fluoroscopy.

Once the needle 126 has been successfully inserted into the epidural space, the syringe 140 is removed. The stylet 124 is inserted into the lead 100 and the sheath 122 is advanced over the lead 100. The sheath 122 is positioned so that its distal end 128 is near or against the distal tip 106 of the lead 100 causing the lead 100 to follow the curvature of the sheath 122. The stylet 124, lead 100 and sheath 122 are then inserted through the needle 126, into the epidural space, as illustrated in FIG. 7.

Referring to FIG. 8, the distal end 132 of the needle 126 is shown passed through the ligamentum flavum L and the assembled sheath 122/lead 100/stylet 124 is shown emerging therefrom. The rigidity of the needle 126 straightens the more flexible sheath 122 as it passes therethrough. However, upon emergence, the sheath 122 is allowed to bend along or toward its precurvature as shown. In some implementations, the shape memory of the sheath 122 material allows the sheath 122 to retain more than 50% of its precurved shape upon passing through the needle 126. Such bending assists in steering of the lead 100 within the epidural space. This is particularly useful when using a retrograde approach to navigate across the transition from the lumbar spine to the sacral spine. The sacrum creates a “shelf” that resists ease of passage into the sacrum. The precurved sheath 122 is able to more easily pass into the sacrum, reducing operating time and patient discomfort.

Referring to FIG. 9, the assembled sheath 122/lead 100/stylet 124 is advanced within the epidural space toward a target DRG. Steering and manipulation is controlled proximally and is assisted by the construction of the assembled components and the precurvature of the sheath 122. In particular, the precurvature of the sheath 122 directs the lead 100 laterally outwardly, away from the midline M of the spinal column. FIG. 10 illustrates the assembled sheath 122/lead 100/stylet 124 advanced toward the target DRG with the precurvature of the sheath 122 directing the lead 100 laterally outwardly.

Referring to FIG. 11, the lead 100/stylet 124 is then advanced beyond the distal end 128 of the sheath 122. The lead 100 may extend, for example, approximately 1-3 inches beyond the distal end 128 of the sheath 122. However, the lead 100 may extend any distance, such as less than 1 inch, 0.25-3 inches, or more than 3 inches. Likewise, the sheath 122 may be retracted to expose the lead 100, with or without advancement of the lead 100. This may be useful when advancement of the lead 100 is restricted, such as by compression of the foraminal opening. The curvature of the stylet 124 within the lead 100 causes the lead 100 to bend further, along this curvature. This allows the laterally directed lead 100 to now curve around toward the target DRG along the nerve root angulation. This two step curvature allows the lead 100 to be successfully steered to position at least one of the electrodes 102 near the target DRG. Embodiments of the present invention, where are described below, can be used to assist in guiding the lead to such a desired position.

The ball shaped distal tip 106 of the lead 100 resists trauma to the anatomy within the spinal column, such as the dural sac, ligaments, blood vessels, and resists imparting trauma to the DRG as the lead 100 is manipulated and advanced into place. Once the distal end 101 of the lead is in its desired position, or is close to its desired position, the sheath 122 and stylet 124 can be removed, leaving the lead 100 in place. However, optionally, the stylet 124 may be left within the lead 100 to stabilize the lead 100, to assist in maintaining position and to resist migration. The DRG may then be stimulated, e.g., using a trial neurostimulation (TNS) device that provides stimulation energy to the at least one electrode 102, as illustrated by energy ring 140 in FIG. 15. It may be appreciated that multiple electrodes may be energized to stimulate the target DRG. It may also be appreciated that the electrodes may be energized prior to removal of the stylet 124 and/or sheath 122, particularly to ascertain the desired positioning of the lead 100. It may further be appreciated that the sheath 122 may be retracted to expose the lead 100 rather than advancing the lead 100 therethrough.

The same needle 126 can then be used to position additional leads within the epidural space. Again, a stylet 124 is inserted into a lead 100 and a sheath 122 is advanced over the lead 100. The sheath 122 is positioned so that its distal end 128 is near or against the distal tip 106 of the lead 100 causing the lead 100 to follow the curvature of the sheath 122. The assembled stylet 124/lead 100/sheath 122 is then inserted through the needle 126, into the epidural space, as illustrated in FIG. 12. The rigidity of the needle 126 straightens the more flexible sheath 122 as it passes therethrough. And, upon emergence, the sheath 122 is allowed to bend along its precurvature as shown. This creates an atraumatic exit of the stylet 124/lead 100/sheath 122 out of the needle 126 since such curvatures resist any directed force into the dura layer of the spinal cord. This also assists in steering of the lead 100 within the epidural space.

Referring to FIG. 13, the assembled sheath 122/lead 100/stylet 124 is advanced within the epidural space toward another or second target DRG. In this example, the second target DRG is on an opposite side of the spinal column from the first target DRG. Again, the precurvature of the sheath 122 can be used to steer the lead 100 and direct the lead 100 laterally outwardly, away from the midline M of the spinal column. Thus, DRGs on each side of the spinal column can be accessed by manipulation of the sheath 122 while entering the epidural space from the same insertion point. FIG. 14 illustrates the assembled sheath 122/lead 100/stylet 124 advanced toward the second target DRG with the precurvature of the sheath 122 directing the lead 100 laterally outwardly.

The lead 100/stylet 124 is then advanced beyond the distal end 128 of the sheath 122. Again, the curvature of the stylet 124 within the lead 100 causes the lead 100 to bend further, along this curvature. This allows the laterally directed lead 100 to now curve around toward the target DRG along the nerve root angulation. This two step curvature allows the lead 100 to be successfully steered to position at least one of the electrodes 102 on, near or about the target DRG. Once desirably positioned, the sheath 122 and stylet 124 are removed leaving the lead 100 in place, as illustrated in FIG. 15. The DRG may then be stimulated by providing stimulation energy to the at least one electrode 102, as illustrated by energy rings 140 in FIG. 15. Again, it may be appreciated that multiple electrodes may be energized to stimulate the target DRG. It may also be appreciated that the electrodes may be energized prior to removal of the stylet 124 and/or sheath 122, particularly to ascertain the desired positioning of the lead 100.

It may be appreciated that any number of leads 100 may be introduced through the same introducing needle 126. For example, the introducing needle 126 can have more than one lumen, such as a double-barreled needle, to allow introduction of leads 100 through separate lumens. Further, any number of introducing needles 126 may be positioned along the spinal column for desired access to the epidural space. For example, a second needle can be placed adjacent to a first needle. This second needle can be used to deliver a second lead to a spinal level adjacent to the spinal level corresponding to the first needle. In some instances, there is a tract in the epidural space and the placement of a first lead may indicate that a second lead may be easily placed through the same tract. Thus, the second needle is placed so that the same epidural tract may be accessed. Alternatively, a second needle is used to assist in stabilizing the tip of a sheath inserted through a first needle. This second needle can be positioned along the spinal column near the target anatomy. As the sheath is advanced, it may use the second needle to buttress against for stability or to assist in directing the sheath. This may be particularly useful when accessing a stenosed foramen which resists access.

FIG. 16 illustrates a plurality of leads 100 positioned within the epidural space, each lead 100 stimulating a different DRG. In this example, the DRGs are on multiple levels and on both sides of the spinal column. The proximal ends of the leads 100 are connected with an IPG (shown in part) which is typically implanted nearby.

Thus, delivery of the lead 100 through the patient anatomy toward a target DRG involves more potential challenges than delivery of conventional spinal cord stimulator leads. For example, one significant challenge is steering the lead 100 within the epidural space, particularly laterally toward the target DRG and curving the lead 100 through the nerve root sleeve angulation to position at least one of the electrodes 106 on, near or about the DRG. In addition, such leads 100 should preferably be atraumatic and resist kinking, migration, fracture or pullout while implanted. Therefore, significant floppiness and flexibility is desired. However, a more flexible lead can be more difficult to manipulate. To overcome these conflicting challenges, a variety of design features have been incorporated into the devices. Leads are often attached to an implanted medical device (IMD), such as an implanted neurostimulator (INS), to deliver electrical stimulation via electrodes of the leads. An IMD often includes a hermetically sealed device housing within which is located electronic circuitry used for generating and controlling the electrical stimulation, and a header which is used to connect the leads to the IMD. The header is often molded from a relatively hard, dielectric, non-conductive polymer and typically has a thickness approximating the thickness of the device housing. The header typically includes a mounting surface that conforms to and is mechanically affixed to a mating sidewall surface of the device housing.

### Guiding Lead Implantation Using Sensed Signals

Now that an exemplary lead 100 and an exemplary delivery system 120 have been described, and exemplary lead implant procedures (sometimes also referred to as delivery methods) have been described, embodiments of the present invention that are for use in guiding implantation of a lead (e.g., the lead 100) to a desired implant position will now be described. Unless stated otherwise, a desired implant position is a position where an electrode of the lead is located near a DRG. In some embodiments, a desired implant position is when two electrodes of a lead are preferably positioned such that the DRG is substantially equidistant between the two electrodes, and thus, two electrodes of the lead are near the DRG. Once in the desired implant position, the lead can then be sutured or otherwise anchored in place to reduce and preferably prevent migration of the lead. Thereafter, a proximal end of the implanted lead can be connected to a non-implanted trial neurostimulator (TNS) or to an INS.

FIG. 17 is a high level flow diagram that is used to summarize various methods for use in guiding implantation of a lead toward an implant position where at least one electrode of the lead is located near a target DRG. For consistency and illustration, references will often be made to the exemplary lead 100 and exemplary delivery system 120 described above with reference to FIGS. 1-16. However, it should be understood that embodiments of the present invention are not limited to use with the exemplary lead 100 and the exemplary delivery system 120.

Referring to FIG. 17, at step 202, a distal end of the lead is inserted into an epidural space of a spinal column within which is located the target DRG. As explained above, e.g., with reference to FIGS. 1-4, the distal end 101 of the lead 100 includes one or more electrodes 102, and in specific embodiments, includes four electrodes 102. Prior to this step, an introducing needle 126 and optionally a syringe 130 will likely be used to access the epidural space, e.g., as was explained above with reference to FIGS. 5 and 6. Thereafter, the distal end 101 of the lead 100 can be inserted through the introducing needle 126 and expelled out the distal tip of the needle, thereby resulting in the distal end 101 of the lead 100 being within the epidural space. As explained above, at this point, at least a portion of the lead 100 may be covered by a sheath 122. Additionally, a stylet 124 can be located within a stylet lumen 104 of the lead 100 such that the stylet 124 extends therethrough so that its distal end 130 aligns with the distal end 101 of the lead 100. As mentioned above, e.g., with reference to FIGS. 8-10, the distal end 101 of the lead 100 can be extended beyond the distal end of the sheath 122 thereby exposing the electrodes 102 to surrounding tissue. This can be achieved by pushing on the proximal end of the lead 100 and/or stylet 124 and thereby advancing the lead. Alternatively, the sheath 122 can be retracted to expose the distal end 101 of the lead 100, with or without advancement of the lead 100.

Referring again to FIG. 17, at step 204, one or more electrode of the lead is used to obtain a sensed signal indicative of proximity of at least one electrode of the lead relative to the target DRG. For example, step 204 can be performed while the distal end of the lead is advanced toward or otherwise maneuvered relative to the target DRG. In certain embodiments, the sensed signal is indicative of a potential difference between two electrodes 102 of the lead 100, and thus, the sensed signal can be referred to as a sensed voltage signal. For example, the potential difference between the two most distal electrodes 102 can be measured. Alternative or additional combinations of the electrodes 102 can be used to sense a voltage signal. It is also within the scope of an embodiment that the sensed voltage signal be obtained by measuring a potential difference between an electrode 102 of the lead 100 and a far-field reference electrode, such as, but not limited to, a patch electrode. In other embodiments, the sensed signal is indicative of an impedance between two electrodes 102 of the lead 100, and thus, the sensed signal can be referred to as a sensed impedance signal. For example, the impedance between the two most distal electrodes 102 can be measured. Alternative or additional combinations of the electrodes 102 can be used to sense an impedance signal. It is also within the scope of an embodiment that the sensed impedance signal be obtained by measuring an impedance between an electrode 102 of the lead 100 and a far-field reference electrode, such as, but not limited to, a patch electrode. It is also within an embodiment to sense both voltage and impedance signals.

In certain embodiments, the signal that is sensed at step 204 is an intrinsic signal, in which case the signal is sensed during a period of time that the patient is not provided with external stimulus. Alternatively, the signal that is sensed at step 204 can be an evoked response signal, in which case the signal is sensed during a period of time that a physical or electrical stimulation is delivered to the targeted body region to induce the evoked response signal that is being sensed. For example, if the DRG on the right side of the spinal level L4 or L5 is being targeted for the purpose of treating right foot pain, then an evoked response signal can be induced by electrically stimulating the patient's right foot using a non-implantable electrical stimulator. Such electrical stimulation can be referred to herein as an evoked response inducing stimulation. Alternatively, to induce an evoked response signal, the patient's right foot can be physically stimulated, e.g., by pricking, pinching, stroking (e.g., with von Frey hairs), or squeezing the patient's right foot, or by touching the patient's right foot with a relatively hot or cold object. These are just a few examples of the types of electrical and physical stimulation that can be delivered to induce an evoked response signal, which are not meant to be all encompassing. Accordingly, other types of electrical and/or physical stimulation can be performed to induce an evoked response signal that can be sensed while still being within the scope of an embodiment of the present invention. A potential benefit of inducing and sensing an evoked response signal is that features of the sensed evoked response signal should occur at specific points in time that can be correlated to the evoked response inducing stimulation to ensure that the system is sensing the appropriate signal. Additionally, the overall amplitude of a sensed evoked response signal may be greater than the overall amplitude of a sensed intrinsic signal, potentially resulting in a higher overall signal-to-noise ratio (SNR), which is desirable.

At step 206, the sensed signal is used to guide placement of at least one electrode of the lead toward an implant position near the target DRG. For example, the amplitude of a sensed voltage signal should increase as an electrode being used to obtain the sensed voltage signal moves closer to the target DRG. This is because the DRG (and attached nerve fibers) produces an electric field that is present in a surrounding area, and the magnitude of this electric field is a function of the distance between the measurement site (i.e., the electrode(s) used to sense the electric field) and the DRG. Explained another way, the magnitude of the electric field produced by the DRG (and attached nerve fibers) generally follows an inverse-square law, and thus, diminishes with the square of the distance away from the DRG. Accordingly, an increase in the sensed voltage signal can be interpreted as an indication that at least one electrode, used to obtain the sensed voltage signal, moved closer to the target DRG. Conversely, a decrease in the sensed voltage signal can be interpreted as an indication that at least one electrode, used to obtained the sensed voltage signal, moved away from the target DRG. Where two electrodes of a lead are being used to obtain the sensed voltage signal, the sensed voltage signal should have its maximum amplitude when the target DRG is equidistant between the two electrodes (used to obtain the sensed voltage signal) and each of the two electrodes is within about 1-3 mm of the target DRG.

As mentioned above, the signal sensed at step 204, and used to guide lead placement at step 206, can be an impedance signal. Dura mater encloses the spinal cord and cerebrospinal fluid (CSF), wherein such CSF has high levels of salinity. The volume of CSF, and thus the salinity level, generally decreases at locations further from the anatomical midline of the spinal cord (medial) and closer to a DRG (lateral) which is surrounded by minimal CSF. Typically, impedance levels are inversely correlated with saline levels. In other words, the lower the salinity, the greater the impedance. This means that the sensed impedance signal should increase as an electrode being used to obtain the impedance signal moves closer to the target DRG. Accordingly, an increase in the sensed impedance signal can be interpreted as an indication that at least one electrode, used to obtain the impedance signal, moved closer to the target DRG.

In still another embodiment, the distal end of the lead can include a chemical sensor that measures saline levels. Since saline levels are very low near a DRG, a signal generated by such a chemical sensor can alternatively, or additionally, be used. Where such a chemical sensor were used, a decrease in the sensed signal indicative of a saline level can be interpreted as an indication that the sensor moved closer to the target DRG. Alternative types of sensors (also referred to as sense elements) can alternatively, or additionally, be used to obtain a sensed signal used to guide lead placement, so long as changes in the sensed signal are indicative of changes in the proximity of the sensor to a target DRG.

In certain embodiments, the signal-to-noise ratio (SNR) of a sensed signal can be determined, and the SNR can be used to guide lead placement at step 206. For example, as mentioned above, where the signal sensed at step 204 is a sensed voltage signal, the sensed voltage signal should increase the closer an electrode (used to obtain the sensed voltage signal) gets to the target DRG. As in many environments, there will likely be background noise that is not of interest (e.g., from sources other than the DRG). Assuming the background noise stays generally constant, and assuming that the signal of interest increases the closer an electrode gets to the target DRG, then it can be appreciated that the SNR should increase the closer an electrode (used to obtain the sensed signal) gets to the target DRG. Accordingly, an increase in the SNR can be interpreted as an indication that at least one electrode, used to obtain the sensed signal, moved closer to the target DRG. The lead is preferably positioned such that stimulation delivered using the lead stimulates the target DRG without stimulating nearby ventral structures. Therefore it may also be the case that by maneuvering the lead more dorsally the background noise from ventral structures is reduced and thereby increases the SNR. Conversely, a decrease in the SNR can be interpreted as an indication that at least one electrode, used to obtain the sensed signal, moved away from the target DRG.

More than one voltage signal and/or more than one impedance signal can be sensed at substantially the same time by sensing voltages (i.e., potential differences) and/or measuring impedances using multiple different combinations of the electrodes 102 of the lead 100 being implanted. For example, where a lead includes four electrodes 102, up to six different electrode pair combinations can be used to sense voltage signals and/or impedance signals. Additionally, or alternatively, where one or more far-field reference electrode(s) is/are used to obtain the sensed signal, sensed signals can be obtained using multiple combinations of electrode(s) 102 of the lead and the far-field reference electrode(s). The sensed signals can be compared to one another, and the signal having the greatest magnitude and/or SNR can be selected and used for guiding at least one of the electrodes toward an implant position near the target DRG. Alternatively, multiple sensed signals can simultaneously be used to guide placement of at least one electrode toward an implant position near the target DRG. For example, information indicative of at least one electrode's position relative to a target DRG can be gleaned from both a sensed voltage signal and a sensed impedance signal, and/or one or more other types of sensed signals.

It is noted that a lead (e.g., 100) can have electrode(s) (e.g., 102) that are for use both as stimulation electrode(s) and sensing electrode(s). For example, particular electrode(s) of a lead can be used for delivering stimulation at one point in time, and for performing sensing at another point in time. Alternatively, a lead can have one or more electrodes that are dedicated for use as stimulation electrodes, and one or more other electrodes that are dedicated for use as sensing electrodes. It is also possible that a device (e.g., an INS or TNS), to which a lead is connected, is capable of delivering stimulation and performing sensing using any electrode(s), or electrode combinations, of the lead. Alternatively, such a device can be configured such that the device uses only certain electrode(s) of a lead for delivering stimulation, and uses only other certain electrode(s) of the lead for performing sensing. In view of the above, it should be understood that the electrode(s) used to obtain a sensed signal at step 204, may, or may not, be the same electrode(s) that will eventually be used to stimulate the target DRG after the lead is finally positioned and chronically (or temporarily) implanted.

In accordance with certain embodiments, at step 206, the sensed signal obtained at step 204 is compared to a predefined threshold. If that threshold is reached, then it is determined that at least one electrode of the lead (used to obtain the sensed signal) is already positioned near the target DRG, and thus, that the lead is ready to be sutured or otherwise anchored in place. In such embodiments, a predefined indicator (e.g., a specific sound, visual indicator and/or vibration) can be used to indicate that the threshold has been reached. Nevertheless, even if the threshold is reached, it would also be possible to maneuver the lead to see if at least one of the electrode of the lead can be moved even nearer to the target DRG.

Preferably, during the lead implant procedure, the patient is instructed to hold as motionless as possible, or is given a local or general anesthesia, to minimize the amount of noise (e.g., signals not of interest) that may be detected from a ventral root or other nearby muscle fibers in the vicinity of the target DRG of from some or other anatomical source.

In the above described embodiments, at least one electrode or other sensor (used to obtain the sensed signal used to guide placement of an electrode of a lead toward the implant position near a target DRG) was described as being part of the lead that was being implanted. Alternatively, or additionally, the sense electrode(s) or other sensor element(s) can be attached to or otherwise be part of the delivery system, e.g., part of the sheath 122.

The above describe techniques can be used to supplement, or potentially replace, imaging techniques (e.g., fluoroscopy) that have conventionally been used to guide the implantation of implantable neuromodulation leads.

### Lead Implant Guidance System

A non-implantable device, such as, but not limited to, a trial neurostimulator (TNS) device, can be used to perform at least some of the steps described with reference to FIG. 17. A TNS device is typically used after the distal end of the neurostimulation lead has been implanted in a patient to test whether the patient responds to neurostimulation, as well as to test various different stimulation parameters. A reason that a TNS device would be a good candidate for performing at least some of the steps described with reference to FIG. 17 is that a TNS already includes connectors configured to be connected to the proximal end of a partially implanted lead. Additionally, many TNS devices already include circuitry that is capable of measuring impedance levels between different pairs of lead electrodes. Further, a TNS device can be designed to include voltage sense circuitry and/or any other circuitry described herein. Where the TNS device is used, the TNS itself can provide indicators to a physician, or other user, to assist in the guidance of lead placement. Alternatively, the TNS can transfer sensed signals to a clinical programmer (e.g., 340 in FIG. 18) or other computing device that can generate indicators to assist with guidance of lead placement. In still other embodiments, an INS can be used to sense signals, and the INS can transfer the sensed signals to a clinical programmer or other computing device that can produce indicators to assist with guidance of lead placement. Alternatively, a device that is dedicated to being used during a lead implantation procedure can be used to perform the steps described with reference to FIG. 17. Such a device can be referred to as a non-implanted lead implant guidance (LIG) device. An example of components that may be included in such an LIG device can be appreciated from the following description of FIG. 18.

FIG. 18 is a high level block diagram that illustrates components of a device 302, according to an embodiment of the present invention, which can be used to assist with guiding lead placement during an implant procedure. The device 302 can be, e.g., an LIG device that is dedicated to being used during an implant procedure. The device 302 can alternatively be a TNS device, or an INS device, but is not limited thereto. In this embodiment, the device 302 is shown as including connectors 304, 306, 308, a switch device 314, voltage sense circuitry 310, impedance measurement circuitry 311, a pulse generator 312, an evoked response inducing stimulation (EVIS) generator 313, a controller 316, memory 318, a communications interface 320, a user interface 322, an audio transducer 324, a visual transducer 316, a tactile transducer 328 and a power supply 330. It would also be possible for some of the components shown in FIG. 18 to be included in a first device, and other components to be included in a second device that is in communication (wired, or wireless) with the first device. For example, an INS or TNS can include certain components, and a clinical programmer in communications with the INS or TNS can include other components. Accordingly, the device 302 can more generally be referred to as a system 302, since it may actually be made up of more than one device. Further, it should be understood that the term system, as used herein, can refer to a system including a single device, or multiple devices.

The connector 304, which includes connector terminals 305, is used to connect the device 302 to a proximal end of the lead 100 that is being implanted. For example, after a distal end 101 of a lead 100 has been inserted into the epidural space of the spinal column within which is located the target DRG, the proximal end of the lead 100 can be connected to the connector 304 of the device 302. In certain embodiments, a lead extension (analogous to an electrical extension cord) can be connected between the switch device 314 and the connector 304, or between the connector 304 and the proximal end of the lead 100. While four terminals 305 are shown, the connector 304 can include more or less than four terminals. It is also possible that there are multiple instances of the connector 304 that enables multiple leads to be connected to the device 302.

The connector 306, which includes connector terminals 307, is used to connect the device 302 to one or more far-field reference electrodes, such as, but not limited to, patch electrode(s) and/or or a metal plate upon which a patient may lie down during an implant procedure. While two terminals 307 are shown, the connector 306 can include more or less than two terminals.

The connector 308, which includes connector terminals 309, is used to connect the ERIS generator 313 of the device 302 to electrodes that are used to deliver evoked response inducing stimulation to a patient. While two terminals 309 are shown, the connector 308 can include more or less than two terminals.

The switch device 314 can include one or more switch array, switch matrix, multiplexer, and/or any other type of switching device suitable to selectively couple selected lead electrodes (e.g., 102) to the voltage sense circuitry 310, the impedance measurement circuitry 311 or the pulse generator 312. The switch device 314 can also be used to selectively couple one or more far-field reference electrodes to the voltage sense circuitry 310, the impedance measurement circuitry 311 or the pulse generator 312. Additionally, the switch device 314 can also be used to selectively couple one or more evoked response inducing electrodes to the ERIS generator 313. While shown as a single block, the switch device 314 can include various different types of switching elements that are appropriate for the type of signals being transferred.

The voltage sense circuitry 310 can include one or more filters, multiplexers, amplifiers and/or analog-to-digital (A/D) converters, as well as other electronic components, as is known in the art. The filter(s) can be used to filter out frequencies that are not of interest from the sensed voltage signals. An exemplary frequency range of interest for complex action potentials can be from about 100 Hz to 10 KHz, however embodiments described herein should not be limited to just this range. The mutliplexer(s) can be used to enable sensed voltage signals of different frequency ranges to be analyzed in a time multiplexed manner. The mutliplexer(s) can also be used to enable multiple voltage signals indicative of the potential differences between multiple different pairs of electrodes to be obtained in a time multiplexed manner. The amplifier(s), which can include programmable gain and/or automatic gain control, can be used to increase the amplitude of the sensed signals. The A/D converter(s) can be used to convert sensed analog signals to digital signals that can be analyzed or otherwise operated on by the controller 316. The voltage sense circuitry 310 can be coupled to specific electrodes 102 of a lead 100 and/or to specific far-field reference electrode(s) via the switch device 314. In accordance with certain embodiments described herein, the voltage sense circuitry 310 is used to sense one or more voltage signal(s) that can be used to guide placement of at least one electrode of a lead toward the implant position near a target DRG.

The impedance measurement circuitry 310 can similarly include one or more filters, multiplexers, amplifiers and/or analog-to-digital (A/D) converters, as well as other electronic components, as is known in the art. The impedance measurement circuitry 311 can be coupled to specific electrodes 102 of a lead 100 and/or to specific far-field reference electrode(s) via the switch device 314. In accordance with certain embodiments described herein, the impedance measurement circuitry 311 is used to sense one or more impedance signal(s) that can be used to guide placement of at least one electrode of a lead toward the implant position near a target DRG.

The controller 316 can include one or more microprocessor, microcontroller, digital signal processor (DSP), application specific integrated circuit (ASIC), field-programmable gate array (FPGA), state machine, or similar discrete and/or integrated logic circuitry. The controller 316 can be used to control the switch device 314, the indicators 322, 324 and 326, and the other components of the device 302. The memory 318 can include RAM, ROM, NVRAM, EEPROM or flash memory, but is not limited thereto. The memory 318 can be used to store software that the controller 316 uses to control the device 302, as well as to store sensed signal data, stimulation parameters, and/or the like.

Where the device 302 is used for sensing but not for delivering neurostimulation, there may be no need for the device 302 to include a pulse generator 312. However, where the device 302 is also capable of being used to test different neurostimulation parameters and/or to electrically stimulate peripheral nerves (e.g., to stimulate a patient's foot to induce an evoked response), then the device 302 will include the pulse generator 312 and/or the ERIS generator 313. The pulse generator 312 can be coupled to specific electrodes 102 of the lead(s) 100 via the switch device 314. The controller 316 can control the pulse generator 312 and/or the ERIS generator 313 to generate stimulation pulses, and control the switch device 314 to couple the stimulation energy to selected electrodes. Exemplary programmable parameters that can be specified include the pulse amplitude, pulse width, pulse rate (also known as repetition rate or frequency), on-periods and off-periods for a stimulation waveform (also known as a stimulation signal).

The power supply 330, which can include a battery, can be used to power the various other components of the device 302. As such, the power supply 330 can be coupled to the switch device 314, the voltage sense circuitry 310, the impedance measurement circuitry 311, the pulse generator 312, the ERIS generator 313, the controller 316, the memory 318, the communications interface 320, the user interface 322, the audio transducer 324, the visual transducer 316 and the tactile transducer 328. A voltage regulator (not shown) can step up or step down a voltage provided by a battery or an external power source to produce one or more predetermined voltages useful for powering such components of the device 302.

The communications interface 320 enables, for example, the device 302 to be connected to general purpose computer, a clinical programmer 340, or other computing device that can be used to program the device 302 display information obtained using the device 302 and/or produce visual, audio and/or tactile indicators. The communications interface 320 can provide for wired and/or wireless communications with such other devices.

The user interface 322 can include a display, a keypad, a touch screen, one or more peripheral pointing devices (e.g., a mouse, touchpad, joystick, trackball, etc.), and/or the like. The controller 316 can provide a graphical user interface (GUI) via the user interface 322 to facilitate interaction with a physician or other user (e.g., a clinician).

The audio transducer 324 can be a speaker or other electroacoustic transducer that can output an adjustable audible sound. The visual transducer 326 can include one or more light emitting elements (e.g., light emitting diodes (LEDs)) or a display that can output an adjustable visual indication. The tactile transducer 328 can include electromechanical components that can output an adjustable vibration. In accordance with specific embodiments of the present invention, one or more of the transducers 324, 326 and 328 can be used to provide feedback, to a physician, that is indicative of how close or far an electrode of the lead being implanted is to the target DRG, as explained below. It is also possible that one or more of elements 322, 324, 326 and/or 328 be part of a further device, such as a clinical programmer 340 or other computing device that communicates with the device 302.

In accordance with an embodiment, the controller 316 controls the audio transducer 324 to produce an audio indicator that changes in dependence on a sensed signal, and thus, is indicative of the proximity of an electrode (used to obtain the sensed signal) relative to the target DRG. For example, the volume of a tone or other sound can be increased to provide an indication that at least one electrode (used to obtain the sensed signal) moved closer to the target DRG. Conversely, the volume of the tone or other sound can be decreased to provide an indication that at least one electrode (used to obtain the sensed signal) moved away from the target DRG. Alternatively, or additionally, the frequency of a tone or other sound can be increased to provide an indication that an electrode moved closer to the target DRG. Conversely, the frequency of the tone or other sound can be decreased to provide an indication that an electrode moved away from the target DRG. For a more specific example, a relatively high pitched sound can indicate that an electrode is close to the target DRG, and a relatively low pitched sound can indicate that an electrode is far from the target DRG. Alternatively, or additionally, the repetition rate of a tone or other sound (e.g., a beeping sound) can be increased to provide an indication that an electrode moved closer to the target DRG. Conversely, the repetition rate of the tone or other sound can be decreased to provide an indication that an electrode moved away from the target DRG. For a more specific example, a relatively fast beeping sound can indicate that an electrode is close to the target DRG, and a relatively slow beeping sound can indicate that an electrode is far from the target DRG.

In accordance with an embodiment, the controller 316 controls the visual transducer 326 to produce a visual indicator that changes in dependence on a sensed signal, and thus, is indicative of the proximity of an electrode (used to obtain the sensed signal) relative to the target DRG. For example, as shown in FIG. 19, the visual transducer 326 can be a multi-LED bar graph display 402 including a plurality of LED segments 404, an example of which is shown in FIG. 19. In such an embodiment, the number of LED segments 404 that are lit (and thus, the height of the displayed bar) can be increased to provide an indication that an electrode moved closer to the target DRG and/or the number of LED segments 404 that are lit can be decreased to provide an indication that the electrode moved away from to the target DRG. For another example, the visual indicator can be a single or multi-LED display that changes color and/or brightness. The color can transition to a green color (or some other selected color) to provide an indication that an electrode moved closer to the target DRG and/or the color can transition to a red color (or some other selected color) to provide an indication that an electrode moved away from to the target DRG. Alternatively, or additionally, an increase in the brightness of the LED(s) can provide an indication that an electrode moved closer to the target DRG and/or a decrease in the brightness can provide an indication that an electrode moved away from to the target DRG.

For still another example, the visual transducer 326 can be a display screen, such as, but not limited to, a liquid crystal display (LCD) screen, a thin film transistor LCD screen (TFT-LCD), a cathode ray tube (CRT) screen, light emitting diode (LED) backlit LCD screen, or an organic light-emitting diode (OLED) screen. Such a display screen can for example display a bar graph or color, that in a similar manner as discussed above, changes height and/or color to indicate whether an electrode is moving closer to, or away from, the target DRG. The display screen can alternatively display textual or other visual indicators to provide a visual indication of the proximity of an electrode to the target DRG. In embodiments where the user interface 322 provides a GUI, the visual transducer 326 can be implemented by the GUI.

In accordance with an embodiment, the controller 316 controls the tactile transducer 328 to produce an vibrating indicator that changes in dependence on a sensed signal, and thus, is indicative of the proximity of an electrode (used to obtain the sensed signal) relative to the target DRG. For example, the magnitude or frequency of vibration can be increased to provide an indication that an electrode moved closer to the target DRG. Conversely, the magnitude or frequency of vibration can be decreased to provide an indication that an electrode moved away from the target DRG.

In certain embodiments, only one of the transducers 322, 324 and 326 are included in the device 302. In other embodiments, more than one of the transducers 322, 324 and 325 are included in the device 302, and a physician can use all of the included transducers, or can select which transducer(s) they want to use. Alternatively, as mentioned above, one or more of the transducers 322, 324 and 325 can be included in a separate device that communicates with the device 302.

In view of the above description, it can be appreciate that a myriad of alternative changes to an audio indicator, a visual indicator and/or a tactile indicator can be used to provide feedback that is indicative of the proximity of an electrode relative to a target DRG.

As explained above, more than one sensed signal (used to guide lead implantation) can be obtained at substantially the same time. For example, multiple voltage signals can be sensed, multiple impedance signals can be sensed, both a voltage signal and an impedance signal can be sensed, or multiple voltage and multiple impedance signals can be sensed. In such embodiments, there can be multiple instances of one of the transducers 322, 324 or 326, so that audio, visual and/or tactile indicators can be produced for more than one sensed signal. For example, a first displayed bar graph can be generated based on a first sensed voltage signal, and a second displayed bar graph can be generated based on a second sensed voltage signal. For another example, a first displayed bar graph can be generated based on a sensed voltage signal, and a second displayed bar graph can be generated based on a sensed impedance signal. For still another example, a displayed bar graph can be generated based on a sensed voltage signal, and an audible indicator can be generated based on a sensed impedance signal. It is also within the scope of an embodiment that a combination of different obtained sensed signals be used to adjust a single indicator. In view of the above description, it can be appreciate that a myriad of combinations of audio, visual and/or tactile indicators can be used to provide feedback that is indicative of the proximity of at least one electrode relative to a target DRG.

Where the communications interface 320 connects the device 302 to a general purpose computer, a clinical programmer 340, or other computing device, one or more of the above described indicators can be generated on the computing device to which the device 302 is connected, e.g., using a display and/or speaker of the computing device.

In the above description, it was generally assumed that the lead being implanted would eventually be used to deliver neurostimulation to the target DRG. However, it should be noted that the lead can additionally, or alternatively, be used to deliver other types of neuromodulation. For example, a lumen of the lead can be used to deliver a pharmaceutical agent to the target DRG to perform neuromodulation. Where this is the case, embodiments of the present invention described above can be used to place an opening or valve in the lead (from which the pharmaceutical agent will be dispensed) near the target DRG. Such an opening or valve can, e.g., be located at the distal tip of the lead or at some other location within the distal end of the lead.

Embodiments of the present invention have been described above with the aid of functional building blocks illustrating the performance of specified functions and relationships thereof. The boundaries of these functional building blocks have sometimes been defined herein for the convenience of the description. Alternate boundaries can be defined so long as the specified functions and relationships thereof are appropriately performed. Any such alternate boundaries are thus within the scope and spirit of the claimed invention.

Although the foregoing invention has been described in some detail by way of illustration and example, for purposes of clarity and understanding, it will be obvious that various alternatives, modifications and equivalents may be used and the above description should not be taken as limiting in scope of the invention.

What is claimed is:

1. A method for use in guiding implantation of a lead toward an implant position where at least one electrode of the lead is located near a target dorsal root ganglion (DRG), the method comprising:
(a) inserting a distal end of the lead into an epidural space of a spinal column within which is located the target DRG;
(b) using at least one electrode of the lead to obtain a sensed signal indicative of proximity of the at least one electrode relative to the target DRG; and
(c) using the sensed signal to guide placement of at least one electrode of the lead toward an implant position near the target DRG.

2. The method of claim 1, wherein step (c) includes:
producing an audio indicator in dependence on the sensed signal, wherein changes in at least one of an amplitude, a frequency or a repetition rate of the audio indicator are indicative of changes in proximity of at least one electrode of the lead relative to the target DRG.

3. The method of claim 1, wherein step (c) includes:
producing a visual indicator in dependence on the sensed signal, wherein changes in the visual indicator are indicative of changes in proximity of at least one electrode of the lead relative to the target DRG.

4. The method of claim 1, wherein step (c) includes:
producing a tactile indicator in dependence on the sensed signal, wherein changes in the tactile indicator are indicative of changes in proximity of at least one electrode of the lead relative to the target DRG.

5. The method of claim 1, wherein the signal that is sensed at step (b), and is used to guide placement of at least one electrode of the lead at step (c), comprises an intrinsic signal.

6. The method of claim 1, wherein the signal that is sensed at step (b), and is used to guide placement of at least one electrode of the lead at step (c), comprises an evoked response signal.

7. The method of claim 6, wherein the lead is for use in stimulating the target DRG to treat pain in a targeted body region that is remote from the target DRG, and wherein the method further comprises:
delivering a physical or electrical stimulation to the targeted body region to induce the evoked response signal that is sensed at step (b) and is used to guide placement of at least one electrode of the lead at step (c).

8. The method of claim 1, wherein
the sensed signal is indicative of a potential difference between an electrode of the lead and a further electrode.

9. The method of claim 8, wherein step (c) includes:
(c.1) interpreting an increase in the sensed signal as an indication that at least one electrode of the lead moved closer to the target DRG; and
(c.2) interpreting a decrease in the sensed signal as an indication that at least one electrode of the lead moved away from the target DRG.

10. The method of claim 8, wherein step (c) includes:
(c.1) determining a signal-to-noise ratio (SNR) of the sensed signal;
(c.2) interpreting an increase in the SNR as an indication that at least one electrode of the lead, used to obtain the sensed signal, moved closer to the target DRG; and (c.3) interpreting a decrease in the SNR as an indication that at least one electrode of the lead, used to obtain the sensed signal, moved away from the target DRG.

11. The method of claim 1, wherein the sensed signal is indicative of an impedance between an electrode of the lead and a further electrode.

12. The method of claim 9, wherein step (c) includes interpreting an increase in the sensed signal as an indication that the electrode moved closer to the target DRG.

13. A system for use in guiding implantation of a lead toward an implant position where an electrode of the lead is located near a target dorsal root ganglion (DRG), the system comprising:
circuitry to obtain a sensed signal from a lead after a distal end of the lead has been inserted into an epidural space of a spinal column within which is located the target DRG; and
a transducer that produces an indicator in dependence on the sensed signal, wherein changes in the indicator are indicative of changes in proximity of at least one electrode of the lead relative to the target DRG.

14. The system of claim 13, further comprising:
a controller to interpret changes in the sensed signal and adjust the indicator so that changes in the indicator are indicative of changes in proximity of at least one electrode of the lead relative to the target DRG.

15. The system of claim 14, wherein:
the transducer comprises an audio transducer that produces an audio indicator; and
the controller controls the audio transducer to change at least one of an amplitude, a frequency or a repetition rate of the audio indicator to indicate changes in proximity of at least one electrode of the lead relative to the target DRG.

16. The system of claim 14, wherein:
the transducer comprises a visual transducer that produces a visual indicator; and
the controller controls the visual transducer to change the visual indicator to indicate changes in proximity of at least one electrode of the lead relative to the target DRG.

17. The system of claim 14, wherein:
the transducer comprises a tactile transducer that produces a tactile indicator; and
the controller controls the tactile transducer to change the tactile indicator to indicate changes in proximity of at least one electrode of the lead relative to the target DRG.

18. The system of claim 14, wherein:
the circuitry to obtain a sensed signal comprises voltage sense circuitry to obtain a sensed signal indicative of a potential difference between an electrode of the lead and a further electrode; and
the controller interprets an increase in the sensed signal as an indication that at least one electrode of the lead, which that was used to obtain the sensed signal, moved closer to the target DRG;
the controller interprets a decrease in the sensed signal as an indication that at least one electrode of the lead, which was used to obtain the sensed signal, moved away from the target DRG; and
the controller adjusts the indicator produced by the transducer in dependence on changes in the sensed signal.

19. The system of claim 14, wherein:
the circuitry to obtain a sensed signal comprises voltage sense circuitry to obtain a sensed signal indicative of a potential difference between an electrode of the lead and a further electrode; and
the controller determines a signal-to-noise ratio (SNR) of the sensed signal;
the controller interprets an increase in the SNR as an indication that at least one electrode of the lead, which was used to obtain the sensed signal, moved closer to the target DRG;
the controller interprets a decrease in the SNR as an indication that at least one electrode of the lead, which was used to obtain the sensed signal, moved away from the target DRG; and
the controller adjusts the indicator produced by the transducer in dependence on changes in the SNR.

20. The system of claim 14, wherein:
the circuitry to obtain a sensed signal comprises impedance measurement circuitry to obtain a sensed signal indicative of an impedance between an electrode of the lead and a further electrode; and
the controller interprets an increase in the sensed impedance signal as an indication that at least one electrode of the lead, which was used to obtain the sensed signal, moved closer to the target DRG; and
the controller adjusts the indicator produced by the transducer in dependence on changes in the sensed signal.

21. A method for use in guiding of a lead toward a position where a sense element of the lead or of a sheath covering at least a portion of the lead is located near a target dorsal root ganglion (DRG), the method comprising:
(a) inserting a distal end of the lead and a distal end of the sheath covering at least a portion of the lead into an epidural space of a spinal column within which is located the target DRG;
(b) using the sense element to obtain a sensed signal indicative of proximity of the sense element relative to the target DRG; and
(c) using the sensed signal to guide positioning of the sense element toward a position near the target dorsal root ganglion (DRG).

22. The method of claim 21, wherein the sense element comprises at least one of an electrode or a chemical sensor.
